(19) United States
(12) Patent Application Publication
(10) Pub. No.: US 2023/0218144 A1
(43) Pub. Date: Jul. 13, 2023

(54) SURGICAL SYSTEM

(71) Applicant: OLYMPUS CORPORATION, Tokyo (JP)

(72) Inventors: Per SUPPA, Hamburg (DE); Dennis BERNHARDT, Hamburg (DE)

(73) Assignee: OLYMPUS CORPORATION, Tokyo (JP)

(21) Appl. No.: 18/093,022

(22) Filed: Jan. 4, 2023

Related U.S. Application Data
(60) Provisional application No. 63/298,300, filed on Jan. 11, 2022.

(51) Int. Cl. A61B 1/00; G06F 3/0354; G06F 3/0338; G06T 19/20; A61B 1/05

(52) U.S. Cl. CPC A61B 1/0004; G06F 3/03549; G06F 3/0338; G06T 19/20; A61B 1/05; A61B 1/00045; A61B 1/00105; G06T 2210/41; G06T 2219/2016

(57) ABSTRACT

A surgical system including a surgical instrument having a shaft, an image sensor positioned at a distal end of the shaft, the image sensor being configured to capture images of a target object of a living object, and a control grip is arranged at a proximal end of the shaft. The surgical system further including a monitor for displaying the images and a controller configured to display a virtual target object model image of the target object on the monitor. Where the control grip is configured to position the virtual target object model image displayed on the monitor at a determined position and rotate the virtual target object model image displayed on the monitor at the determined position.

### CROSS-REFERENCE TO RELATED APPLICATION

The present application is based upon and claims the benefit of priority from U.S. Provisional Patent Application No. 63/298,300 filed on Jan. 11, 2022, the entire contents of which is incorporated herein by reference.

### BACKGROUND

### Field

The present disclosure relates to a surgical system and a method for operating a surgical system. Moreover, the present disclosure further relates to an adapter device for a control grip of the surgical instrument, such as, an endoscope or a laparoscope.

### Prior Art

Surgeries with a laparoscope, also known as laparoscopic surgeries or laparoscopies, offer a number of advantages compared to traditional open surgeries, e.g., smaller incisions and a shorter recovery time. In laparoscopic surgery, intraoperative navigation plays a crucial role. However, there are a number of difficulties involved in this kind of navigation. The organ cannot be touched, so that haptic feedback is unavailable to the surgeon. Thus, the position of a tumour cannot be felt. In addition, laparoscopic ultrasound is the only available technique to visualize sub-surface structures, e.g., veins, arteries and tumours, under the surface of the target organ. Laparoscopic ultrasound usually only provides 2D images which need to be integrated in the surgeon's mind to refer to the actual position of the 2D ultrasound slice in 3D space.

In laparoscopic surgery, the surgeon gets the visualization of the intraabdominal surgical field on a 2D monitor. With the availability of preoperative CT (computer tomography) and MRI (magnetic resonance image) 3D scan technology, it became possible to reconstruct 2D radiological imaging slices into a virtual 3D model. Moreover, the virtual 3D model can be printed with modern 3D printer technology and can be used by the surgeon during the procedure to aid in spatial navigation and to assess the distance of risk structures relative to each other. Another commonly used approach is to directly use the virtual 3D model in the OR by implementing a software application that displays the model.

An object is to improve the usability of a surgical system in an easy, fast, reliable and inexpensive way.

Such object can be solved by a surgical system comprising

a surgical instrument having a shaft with a distal end and a proximal end, wherein an image sensor is positioned at the distal end of the shaft and a control grip is arranged at the proximal end of the shaft for controlling at least one function of the surgical instrument,

a monitor for displaying images of a target object of a living object, wherein the images are captured by the image sensor of the surgical instrument,

a means for providing a virtual target object model image, such as a 3D virtual target object model image, of the target object and for displaying the virtual target object model image of the target object,

wherein the control grip is provided with positioning means for positioning the virtual target object model image on the monitor at a determined position and wherein the control grip is provided with rotation means for rotating the virtual target object model image at the determined position of the virtual target object model image in the monitor.

During a surgery with the surgical system, the virtual target model image of a target object is simultaneously and directly displayed on the monitor or screen of the surgical system, which displays the images of the target object, captured by the image sensor of the surgical instrument. During the procedure a surgeon can manipulate the displayed virtual target model image, such as three-dimensional target model image, of the target object by operating the positioning means and/or the rotation means. The positioning means can be configured to move a frame of the virtual target model image in a horizontal and/or a vertical direction on the monitor. Accordingly, the frame of the virtual target model image is moved, such as manually, by operation of the positioning means. Furthermore, the rotation means is configured to rotate the displayed virtual target model image around a center of the frame of the virtual target model image in the three dimensions by an operation of the rotation means.

Accordingly, a surgeon can manipulate the virtual target model image, such as the 3D target model image, in order to display relevant information (e.g., show certain vessels only, apply transparency to surfaces, etc.). Further, a surgeon can move the virtual target model image on the monitor in order to overlay the virtual target model image onto the captured image of the target object of the living object. Thereby, it is possible to visualize subsurface structures of the target object. In addition, it is possible for the surgeon to display further or additional information, extracted from a preoperative planning phase (e.g., displaying planned resection plane for intraoperative resection guidance).

According to an embodiment, the positioning means can comprise at least one operable, button. The positioning means can also comprise several manually operable buttons for positioning the virtual target object model image or its frame at the determined position on the monitor.

Moreover, the positioning means can comprise a pair of buttons for a horizontal movement of the virtual target object model image on the monitor. For example, by manually pressing one button, the virtual target object model image or its frame is moved in one horizontal direction on the monitor. By manually pressing the other button, the virtual target object image are moved in the other horizontal direction.

The positioning means can comprise a pair of buttons for a vertical movement of the virtual target object model image on the monitor. By pressing one button for a vertical movement the virtual target model image, such as the virtual 3D target model image, or its frame are moved in the upward direction, while by pressing the second button the virtual target model image or its frame are moved in the downward direction.

Pursuant to an embodiment of the surgical system, the positioning means can be configured as a joystick. By using the joystick, the virtual target object model image or its frame are moved on the monitor corresponding to the movements of the joystick. The joystick can be operated manually by a surgeon during the procedure.

The rotation means can be configured as a free rotatable trackball. Thereby the virtual target object model image allows six degrees of freedom motion of the virtual target object model image in a three-dimensional space.

Furthermore, in an embodiment of the surgical system, the positioning means and the rotation means can be provided on an adapter device. The adapter device can be positioned on the control grip of the surgical instrument. Thereby, for example a surgeon can operate the surgical instrument and the movement of the virtual target object model image with one hand.

The adapter device can be attachably arranged on the control grip of the surgical instrument. That is, the adapter device can be attached to the control grip or can easily be removed from the control grip.

In one embodiment of the surgical system, the adapter device can have a fastener such as a clamp, Velcro (hook and loop) strap etc. for fastening the adapter device onto the control grip.

According to another aspect, the surgical instrument can be configured as an endoscope or as a laparoscope.

The means for providing and displaying the virtual target object model of the target object can be configured as a computer or otherwise, a processor, controller or CPU.

Such object can be solved by a method for operating a surgical system as described above. In order to avoid unnecessary repetitions reference is expressly made to the above explanations.

Furthermore, such object can be solved by an adapter device for a control grip of the surgical instrument, such as an endoscope or a laparoscope, wherein the adapter device comprising positioning means for positioning a virtual target object model image, such as a 3D virtual target object model image, of a target object, such as of a living object, on a monitor of a surgical system at a determined position and further comprising rotation means for rotating the virtual target object model image at the determined position of the virtual target object model image on the monitor.

The positioning means can comprise at least one operable or manually operable, button.

According to another embodiment, the positioning means can comprise a pair of buttons for a horizontal movement of the virtual target object model image on the monitor.

In another embodiment, the positioning means can comprise a pair of buttons for a vertical movement of the virtual target object model image on the monitor.

In an alternative of the adapter device, the positioning means can be configured as a joystick.

In addition, the rotation means can be configured as a free rotatable trackball.

The positioning means and the rotation means can be provided on the adapter device.

An embodiment of the adapter device includes the adapter device being attachably arranged on the control grip of the surgical instrument.

The adapter device can have fastener for fastening the adapter device onto the control grip.

Moreover, such object can be solved by the use of an adapter device, as described above in a surgical system, as explained in the above paragraphs.

### BRIEF DESCRIPTION OF THE DRAWINGS

Further characteristics will become apparent from the description of the embodiments according together with the claims and the included drawings. Embodiments can fulfill individual characteristics or a combination of several characteristics.

The embodiments are described below, without restricting the general intent of the invention, based on exemplary embodiments, wherein reference is made expressly to the drawings with regard to the disclosure of all details that are not explained in greater detail in the text. The drawings show in:

FIG. 1 schematically illustrates a surgical system;

FIG. 2a schematically illustrates a view of a handle grip of the endoscope;

FIG. 2b schematically illustrates a cross section of the handle grip; and

FIG. 3 schematically illustrates a cross-section of the handle grip according to another embodiment.

In the drawings, the same or similar types of elements or respectively corresponding parts are provided with the same reference numbers in order to prevent the item from needing to be reintroduced.

### DETAILED DESCRIPTION

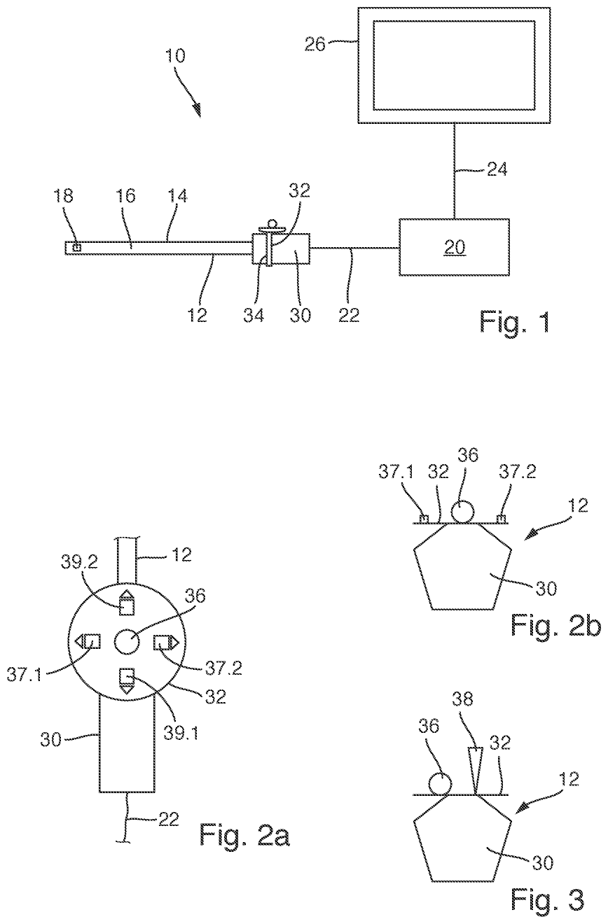

FIG. 1 shows schematically a structure of a surgical system 10, which comprises a video endoscope 12 and control unit 20, such as a controller, processor, CPU or general purpose computer. The control unit 20 is connected to the video endoscope 12 via a cable 22. The video endoscope 12 has a hermetically sealed housing 14 with an elongated endoscope shaft 16 having an image sensor 18 at its distal end. In addition, the video endoscope 12 has other electrically operated units (not shown here) inside the housing 14 or endoscope shaft 16, such as sensors, actuators, etc.

At its proximal end, the video endoscope 12 has a handle grip 30 which is connected to one end of the cable 20. Furthermore, at its end facing away from the handle 18 of the video endoscope 12. The video endoscope 12 can be configured as a video laparoscope.

The control unit 20 is further connected wire a cable 24 with monitor 26 in order to display the images captured by the image sensor 18. The image sensor 18 can capture images of a (not shown) target object, which is inspected or investigated with the endoscope 20. Moreover, the handle grip 30 is equipped with an adapter 32, which is provided with positioning means and rotation means for moving a virtual target object model image of a target object, which is inspected by the endoscope 12.

A schematic embodiment of the adapter 32 is shown in a top view in FIG. 2a, while FIG. 2b shows a cross-section through the handle grip 30 and the adapter 32. The adapter 32 can be attached to the handle grip 30 by a fastener 34 (see FIG. 1). Configurations of fasteners used for fastening items to a handle are numerous and well known in the art, including a clamp, a band wrapping around the handle and any known fastener for securing/tightening such band in position on the handle, such as a Velcro strap and other fasteners that permits attaching and detaching the adapter to/from handle. On the top the adapter 32 comprises a trackball 36 in the center configured to rotate in any direction abouts its spherical center. The trackball 36 is surrounded by a pair of buttons 37.1, 37.2 and a second pair of buttons 39.1, 39.2. The buttons 37.1, 37.2 are used to move a provided virtual target object model image, such as a three-dimensional virtual target object model image, or is frame in opposing horizontal directions on the monitor 26. The buttons 39.1, 39.2 are provided to move the virtual target model image or its frame in opposing vertical directions on the monitor 26.

The images of a target object are captured by the image sensor 18 and are displayed on the monitor 26 by the control unit 20. Simultaneously, a corresponding virtual target model image of the target object is also displayed on the monitor 26. Display of such images on a monitor by a control unit are well known in the art. After positioning the virtual target model image on the monitor 26 in the horizontal and/or the vertical direction, the virtual target model image can be freely rotated by operation of the trackball 36. The trackball 36 can be freely rotated around all axes by providing a precise movement of a (virtual) 3D model in a three-dimensional space.

Another embodiment of an adapter 32 is shown in FIG. 3, which illustrates a schematic cross-section through the grip handle 30. The adapter 32, also provided with the fastener 34, is provided with a joystick 38 for moving the virtual target object model image in the horizontal direction and in the vertical direction on the monitor 26, while the trackball 36 is provided for rotating the virtual target model image at the position of the virtual target object model image on the monitor 26.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCES

10 Surgical system
12 Video endoscope
14 Housing
16 Endoscope shaft
18 Image sensor
20 Control unit
22 Cable
24 Cable
26 Monitor
30 Handle grip
32 Adapter
34 Fastener
36 Trackball
37.1, 37.2 Button
38 Joystick
39.1, 39.2 Button

What is claimed is:

1. A surgical system comprising:
a surgical instrument comprising:
a shaft with a distal end and a proximal end,
an image sensor positioned at the distal end of the shaft, the image sensor being configured to capture images of a target object of a living object, and
a control grip is arranged at the proximal end of the shaft for controlling at least one function of the surgical instrument,
a monitor for displaying the images,
a controller comprising hardware, the controller being configured to display a virtual target object model image of the target object on the monitor,
wherein the control grip comprises:
positioning means for positioning the virtual target object model image displayed on the monitor at a determined position, and
rotation means for rotating the virtual target object model image displayed on the monitor at the determined position.

2. The surgical system according to claim 1, wherein the positioning means comprises at least one button.

3. The surgical system according to claim 2, wherein the at least one button comprises a pair of buttons for controlling a horizontal movement of the virtual target object model image displayed on the monitor.

4. The surgical system according to claim 2, wherein the at least one button comprises a pair of buttons for controlling a vertical movement of the virtual target object model image displayed on the monitor.

5. The surgical system according to claim 1, wherein the positioning means comprises a joystick (38).

6. The surgical system according to claim 1, wherein the rotation means comprises a trackball.

7. The surgical system according to claim 1, wherein the positioning means and the rotation means are provided on an adapter device connected to the control grip.

8. The surgical system according to claim 7, wherein the adapter device (is configured to be attachably connected on the control grip.

9. The surgical system according to claim 7, wherein the adapter device comprises a fastener configured to fasten the adapter device onto the control grip.

10. The surgical system according to claim 1, wherein the surgical instrument is configured as an endoscope.

11. The surgical system according to claim 1, wherein the controller is configured to display the virtual target object model image as a 3-D virtual target object model image.

12. A method for operating a surgical system comprising:
displaying images of a target image on a monitor, the images being captured by an image sensor positioned at the distal end of the shaft of a surgical instrument;
displaying a virtual target object model image of the target object on the monitor,
positioning the virtual target object model image displayed on the monitor at a determined position from a control grip of the surgical instrument, and
rotating the virtual target object model image displayed on the monitor at the determined position from the control grip of the surgical instrument.

13. An adapter device for a control grip of a surgical instrument, the adapter device comprising:
positioning means for positioning a virtual target object model image of a target object displayed on a monitor of a surgical system at a determined position;
rotation means for rotating the virtual target object model image displayed on the monitor at the determined position.

14. The adapter device according to claim 13, wherein the positioning means comprises at least one button.

15. The adapter device according to claim 14, wherein the at least one button comprises a pair of buttons for controlling a horizontal movement of the virtual target object model image displayed on the monitor.

16. The adapter device according to claim 14, wherein the at least one button comprises a pair of buttons for controlling a vertical movement of the virtual target object model image displayed on the monitor (26).

17. The adapter device according to claim 13, wherein the positioning means comprises a joystick (38).

18. The adapter device according to claim 13, wherein the rotation means comprises a trackball.

19. The adapter device according to claim 13, further comprising a fastener for fastening the adapter device onto the control grip.

20. A control apparatus for operating a surgical system, the control apparatus comprising:
a controller comprising hardware, the controller being configured to:
display images of a target image on a monitor, the images being captured by an image sensor positioned at the distal end of the shaft of a surgical instrument;
display a virtual target object model image of the target object on the monitor,
position the virtual target object model image displayed on the monitor at a determined position from a first input entered on the control grip of the surgical instrument, and
rotate the virtual target object model image displayed on the monitor at the determined position from a second input entered on the control grip of the surgical instrument.
